(19) United States
(12) Patent Application Publication
(10) Pub. No.: US 2024/0082153 A1
(43) Pub. Date: Mar. 14, 2024

(54) IVERMECTIN FORMULATION IN ORAL SOLUTION

(71) Applicant: PROCAPS S.A., Barranquilla (CO)

(72) Inventors: Carlos Manuel Padilla Botett, Barranquilla (CO); Diego Rafael Monterroza Hernandez, Barranquilla (CO); Janice Garcia Vera, Barranquilla (CO); Jose Alfredo Acosta Urrea, Barranquilla (CO); Alfredo Berthel Beleño, Barranquilla (CO)

(21) Appl. No.: 18/270,518

(22) PCT Filed: Dec. 30, 2020
(86) PCT No.: PCT/IB2020/062551

§ 371 Date: Jun. 30, 2023

(51) Int. Cl. A61K 9/08; A61K 31/7048; A61K 47/10; A61K 47/12; A61K 47/14; A61K 47/22; A61K 47/44

(52) U.S. Cl. CPC A61K 9/08; A61K 31/7048; A61K 47/10; A61K 47/12; A61K 47/14; A61K 47/22; A61K 47/44

(57) ABSTRACT

The present invention relates to a formulation of Ivermectin in oral solution which allows proper absorption, enabling the drug to be consumed at any time. In particular, the oral lipid composition is designed with the appropriate selection of a medium-chain triglyceride oil, a hydrophilic cosolvent and an antioxidant, so as to influence the absorption/digestibility of the poorly water-soluble active ingredient and facilitate bioavailability and bioactivity for the administration of the oral solution in drops. The formulation includes Ivermectin in a lipid medium including a cosolvent and an antioxidant in combination with an oily solvent to form an oral solution for administration in drops. The field of the invention is related to improving the bioavailability of insoluble drugs such as Ivermectin for administration in oral drop forms based mainly on lipids without compromising stability or creating impurities.

### FIELD OF THE INVENTION

The present invention refers to a formulation of Ivermectin in oral solution for delivery via a dropper. Particularly, the present invention refers to a formulation of Ivermectin in oral solution for administration in drops, which allows correct absorption in aqueous gastrointestinal media, making it possible for the drug to be consumed at any time.

Ivermectin, as an insoluble active, according to the invention can be formulated in a liquid solution without compromising its stability and without the formation of impurities. Thus, the invention comprises a formulation that includes Ivermectin in a lipid medium that includes a co-solvent and an antioxidant in combination with an oily solvent to form an oral drop-delivery solution.

The field of the invention is related to improving the bioavailability of insoluble drugs such as Ivermectin for their delivery in oral drop solution forms.

### BACKGROUND OF THE INVENTION

The Ivermectin molecule is complex and difficult to formulate. This is due to its physicochemical characteristics. Ivermectin is subject to degradation by hydrolysis, oxidation, acid medium, alkaline medium, light and temperature and accordingly the invention is to design a stable composition in solution for oral administration in drops.

Ivermectin is useful for controlling and treating a broad spectrum of infections caused by parasitic nematodes (roundworms) and arthropods (insects, ticks, and mites) that affect livestock and domestic animals.

The effects of these types of parasites can be serious. For example, ticks are responsible for the transmission and spread of many human and animal diseases throughout the world. The most economically important ticks include Boophilus, Rhipicephalus, Ixodes, Hyalomma, Amblyomma, and Dermacentor. They are vectors of bacterial, viral, rickettsian, and protozoal diseases, and cause tick paralysis and toxicosis. Even a single tick can cause paralysis by injecting its saliva into its host during the feeding process. Tick-borne diseases are generally transmitted by ticks from multiple hosts. These diseases, including babesiosis, anaplasmosis, theileriosis, and heartwater, are responsible for the death and/or debilitation of large numbers of domestic and food animals throughout the world. In many temperate countries, Ixodid ticks transmit the agent of a chronic debilitating disease, Lyme disease, from wildlife to man.

In addition to disease transmission, ticks are responsible for large economic losses in livestock production. Losses are attributable not only to death, but also to skin damage, growth loss, reduced milk production, and reduced meat quality.

Although Ivermectin is an FDA-approved broad-spectrum antiparasitic agent (González Canga et al., 2008), researchers at the Biomedical Discovery Institute at Monash University Melbourne have in recent years shown that Ivermectin possesses antiviral activity against a wide range of viruses (Gotz et al., 2016; Lundberg et al., 2013; Tay et al., 2013; Wagstaff et al., 2012) in vitro.

Originally identified as an inhibitor of interaction between the integrase protein (IN) the human immunodeficiency virus-1 (HIV-1) and the heterodimer a/pi importin (IMP) responsible for the nuclear import of IN (Wagstaff et al., 2011), ivermectin has since been confirmed to inhibit IN nuclear import and HIV-1 replication (Wagstaff et al., 2012). Other actions of ivermectin have been reported (Mastrangelo et al., 2012), but ivermectin has been shown to inhibit host nuclear import (p. (Kosyna et al., 2015; van der Watt et al., 2016)) and viruses, proteins, including simian virus SV40 large tumor antigen (T-ag) and virus non-structural protein 5 (DENV) (Wagstaff et al., 2012, Wagstaff et al., 2011). Importantly, it has been shown to limit infection by RNA viruses such as DENV 1-4 (Tay et al., 2013), West Nile Virus (Yang et al., 2020), Venezuelan Equine Encephalitis Virus (VEEV) (Lundberg et al., 2013), and influenza (Gotz et al., 2016), and this broad-spectrum activity is thought to be due to the reliance of many different RNA viruses on IMPa/pi during infection (Caly et al., 2012; Jans et al., 2019).

Similarly, ivermectin has been shown to be effective against pseudorabies DNA virus (PRV) both in vitro and in vivo, and ivermectin treatment increases survival in PRV-infected mice (Lv et al., 2018). Ivermectin has also been shown to have antiviral activity against the causative agent of the current COVID-19 pandemic, SARS-CoV-2, which is a single-stranded, positive-sense RNA virus that is closely related to the respiratory syndrome coronavirus, severe acute (SARS-CoV). Studies on SARS-CoV proteins have revealed a potential role of IMPa/pi during infection in signal-dependent nucleocytoplasmic closure of the SARS-CoV nucleocapsid protein (Rowland et al., 2005; Timani et al., 2005; Wulan et al., 2015), which can affect host cell division (Hiscox et al., 2001; Wurm et al., 2001). In addition, the SARS-CoV accessory protein ORF6 has been shown to antagonize the antiviral activity of the transcription factor.

STAT1 sequestering IMPa/p1 in the rough ER/Golgi membrane (Frieman et al., 2007). Taken together, these reports suggested that Ivermectin's nuclear transport inhibitory activity may be effective against SARS-CoV-2 in vitro, causing an approximately 5,000-fold reduction in viral RNA after 48 hours.

According to said study, Ivermectin binds to the lmpa/01 heterodimer and destabilizes it, preventing Impa/1 from binding to the viral protein and preventing it from entering the nucleus. This likely results in reduced inhibition of antiviral responses, leading to a normal and more efficient antiviral response.

All this scenario reveals new treatment scenarios associated with the use of Ivermectin as an effective drug in multiple pathologies.

Ivermectin is a slightly hygroscopic, white crystalline powder which is practically insoluble in water. Therefore, it presents a significant challenge to improve the bioavailability of insoluble drugs such as Ivermectin through oral, topical, solid and injectable delivery systems.

It is known within the state of the art that the intake of drugs that incorporate Ivermectin in aqueous solutions should not be administered concomitantly with food, since its absorption and therefore its activity on the target of action is hindered. The technique reports that Ivermectin is administered orally through aqueous solutions, tablets, and some topical creams are also reported.

The prior art reveals injectable liquid forms such as the one referenced in U.S. Pat. No. 5,788,978 to Passeron et al., where an injectable Ivermectin composition is defined that has a programmable release rate and that provides multiple active Ivermectin concentration peaks to produce a pulsed sequence of Ivermectin release in the blood of cattle and horses.

The composition comprises a 0.2%-10% w/w solution of Ivermectin in a solvent selected from propylene glycol and a mixture of glyceryl caprylate, caproate and caprate, such as glycerides of caproic, caprylic and capric acids in equal parts.

The solution is used as a vehicle to suspend microspheres 100 nm-200 pm in diameter of a degradable polymer containing between 0.5% and 50% Ivermectin. The microspheres can be formed from polylactic acid, polyglycolic acid, or a polylactic-polyglycolic acid copolymer. The multi-pulse programmable release system can also be obtained with a biodegradable matrix selected from hardenable natural polymers, such as gelatin or albumin, as well as lactic and glycolic acid copolymers. Polymers can be subjected to a hardening process to increase resistance to biological agents, eg glutaraldehyde solution or alum, or by heating the proteins to coagulation temperature. In one embodiment, Ivermectin-loaded gelatin microspheres are treated in a 25% aqueous glutaraldehyde solution for 24 hours and then suspended in the solvent.

Another embodiment includes a suspension of microspheres loaded with Ivermectin in 1:1 DL-lactic-glycolic copolymer. This ratio of monomers can be modified to improve erosion resistance. However, this liquid form cannot be encapsulated due to the excipients used, as well as the surfactants and co-surfactants, where as it is expressed it is poorly absorbed and therefore has low bioavailability in the digestive system.

U.S. Pat. No. 7,754,696 to Strobel Michael, illustrates a stable and pleasant solution of Ivermectin in water for mass medication of animals. The present formulation does not require the use of benzyl alcohol and is stable indefinitely in concentrated form and up to 30 days when mixed with water.

In contrast to what has been previously described, it is evident that the state of the art does not reveal compositions in solution for administration as drops that incorporate Ivermectma. The foregoing fulfills a long felt need due to the fact that the active ingredient Ivermectin presents great formulation challenges as it is a complex molecule, whose physicochemical characteristics mean that it is easily degradable by hydrolysis, oxidation, acid medium, alkaline medium, light and temperature; with which drawbacks are generated in terms of the stability of the product and solubility; with a related effect on bioavailability.

The present invention is incorporated as an optimal alternative for the administration of Ivermectin that allows accuracy in the dosage and adherence to the treatment by the patient.

Likewise, the present invention incorporates an oral solution for the delivery of Ivermectin drops that is effective in terms of rapid absorption and easy swallowing, with good solubility of the active ingredient, controlling product stability challenges and increasing bioavailability.

### OBJECT OF THE INVENTION

Therefore, a first object of the present invention is to avoid the drawbacks of the prior art. Particularly, the main object of the present invention is to create a formulation of Ivermectin as an insoluble active in a liquid solution with effective bioavailability.

According to the present invention, the main object of the invention is to create an Ivermectin formulation in a lipid medium to improve release and solubility and therefore increase its bioavailability.

It is of paramount importance for formulation scientists to explore the potential of a solution delivery system by combining appropriate excipients based on critical parameters such as surfactant concentration, oil/surfactant ratio, fine oil droplet size, and compatibility, which allows it to be administered as a unitary form for administration orally.

Another important object is the mechanisms involved to improve the solubility and bioavailability of the active without compromising stability and therefore the creation of impurities.

The present invention meets these needs and provides other related advantages. The novel features which are considered to be the basis of the invention are set forth in particular in the appended claims and the additional advantages thereof, will be better understood from the following detailed description with the preferred embodiments.

### DETAILED DESCRIPTION OF THE INVENTION

The present invention refers to a formulation of Ivermectin in solution for oral administration. Particularly, the present invention refers to a formulation of Ivermectin as an insoluble active in a liquid solution with optimal stability and without the formation or presence of impurities suitable for delivery in drops.

The invention is related to improving the bioavailability of insoluble drugs such as Ivermectin for their delivery in oral drop forms. Particularly, the active principle of Ivermectin is complicated and difficult to formulate, because it is affected by both the internal and external environment, such as light, heat, humidity, and gastrointestinal acids further adding to the fact that it is a molecule insoluble in water.

For this purpose, the formulation of the invention is designed by means of a lipid-based drug delivery system whose excipients control the formation of impurities and their choice defines their release and solubility for effective bioavailability. Consequently, the invention was developed from a system that uses a micro emulsion achieved by chemical means.

A lipid-based active delivery system without surfactant, consisting of an oily vehicle, a hydrophilic cosolvent, and an antioxidant, is selected to influence the overall solubility of the poorly soluble active and promote its bioavailability. This in addition to sweetener and flavoring.

The formulation according to the invention comprises an insoluble active such as Ivermectin in combination with excipients such as a solvent, a sweetener, a flavoring, an antioxidant and an oily vehicle based on medium chain triglycerides.

According to the present invention, the composition comprises the use of medium chain triglycerides in a proportion of between 80 and 90%, preferably up to 87% of the composition. In this regard, the inventors have found that medium chain triglycerides are excellent solubilizers for the active ingredient.

The oily phase which is 95% medium chain triglycerides, is selected for its role in the solubilization of Ivermectin and its high polarity and release. Based on the invention, it has been found that medium-chain fatty acid triglycerides solubilize in water faster than long-chain fatty acid triglycerides, and this is measured by a laboratory-level assay (EHM) for hydrolysis using NaOH for hydrolysis. The EHM of medium chain fatty acid triglycerides is 14.9 ml NaOH compared to other triglycerides that require less (higher titrant consumption=higher degree of hydrolysis/lipolysis). The oily phase can be chosen from a medium chain triglyceride (MCT) made up of caprylic/capric/lauric fatty acids.

The oil phase as the important ingredient in the formulation are glycerol fatty acid esters containing 8 to 12 carbon atoms and are selected for their high solvent capacity of Ivermectin and this capacity is mainly decided by the effective concentration of groups ester and for being less prone to oxidation due to the absence of unsaturated acids.

Additionally, the incorporation of an antioxidant to the formulation proposed in the present invention prevents or controls the oxidation of the components present, especially the oils whose physicochemical characteristics make them more susceptible to degradation.

The formulation according to the invention comprises an insoluble active such as Ivermectin in combination with excipients such as an oily solvent, sweetener, flavoring, anti-oxidant and solvent based on medium chain triglycerides.

Particularly, the formulation comprises a liquid solution comprising Ivermectin in an amount of 0.5 to 1.2%, preferably 0.6% of the composition.

The vehicle according to the present invention comprises an oily solvent based on medium chain triglycerides present in the composition in an amount of between 80 and 90%, preferably in the order of 87%.

The solvent is present in the composition in an amount of between 10% and 20%, preferably around 14% and an amount of 0.00001% to 0.1% of an antioxidant.

The sweetener may comprise an amount between 0.5 and 1% and the flavoring may comprise between 1 and 2%. For its part, the sweetener can be selected, for example, from sucralose.

Among the oily solvents selected for the composition of the present invention, it can be selected from canola oil, corn oil, cottonseed oil, sesame oil and soybean oil. It can also be based on oily solvents from CAPTEX™300, Medium Chain Triglycerides (MCT) such as Miglyol™ 810 (Caprylic/Capric Triglyceride) and Miglyol™ 812 (extracts from endosperms of palm oil and or coconut plants).

The solvents selected for the present invention comprise, for example, a rectified alcohol, with a minimum purity of 99.6% v/v, such as anhydrous ethyl alcohol.

The antioxidant is selected from Butylated Hydroxyamsol (BHA) Butylated Hydroxytoluene (BHT), Propylgallate, Sesamol, Ascorbic Acid, Ascorbyl Palmitate, Malic Acid, Sodium Ascorbate, Sodium Metabisulfite, Tocopherol, and DL-Alpha-Tocopherol.

In relation to digestibility, the oily vehicle that integrates the oral solution in drops, contains the active ingredient and allows through the glycerol medium chain fatty acids (MCFAs=C₈: 50-80%-C₁₀: 20-50%-C₁₂: 3%=chain with a length of 8 to 12 C atoms), to generate greater digestibility to guarantee adequate bioaccessibility of the bioactive compound.

Regarding hydrolysis, medium chain triglycerides are fatty acid esters of glycerol are rapidly hydrolyzed. Due to their smaller molar mass (shorter chain lengths), they do not require the formation of chylomicrons for their uptake and transport (EHM=Estimated Hydrolysis Maximum: 14.95 ml of NaOH consumed in vitro in the medium chain triglyceride lipolysis assay.

It was also found that, in relation to lipolysis, the half-life in seconds of medium chain triglycerides is 3349 compared to 715 seconds for mono-diglycerides of medium chain fatty acids (mainly capric).

Regarding digestion and absorption, M-medium-chain triglycerides are fatty acid esters of glycerol and are digested more rapidly and the released medium-chain fatty acids (MCFAs) are absorbed directly into the bloodstream through the portal system of the arteries, and intestinal microvilli because they are soluble in water. Of note, medium chain triglycerides do not stimulate gastrointestinal hormones and therefore do not require bile or pancreatic enzyme, do not require micelle formation prior to absorption, and are absorbed into the albumin-bound portal circulation. They do not particularly require carnitine for transport to the mitochondrion and they are poorly stored in adipose tissues. The plasma half-life is approximately 17 minutes, compared to 33 minutes for long-chain triglycerides and other types of glycerides.

Therefore, based on the invention, the oral lipid composition is designed with the the proper selection of a medium-chain triglyceride oil, a hydrophilic cosolvent and an antioxidant, to influence the absorption/digestibility of the poorly water-soluble active and facilitate the absorption, bioavailability and bioactivity for the administration of the oral solution in drops.

Mainly, the formulation of Ivermectin in oral solution comprises Ivermectin in an amount of between 0.5 to 1.2% by weight of the formula; an amount of oily solvents based on medium chain triglycerides in an amount between 80 to 90%, preferably 87%; a co-solvent in a proportion of 10 to 20%, preferably 14%; an antioxidant in an amount of 0.00001% to 0.1%; and, flavorings and sweeteners to complete the formulation.

### Example 1

According to the present invention, an example of an Ivermectin formulation in oral solution for administration in drops was provided, which allows correct absorption in aqueous gastrointestinal media, allowing the drug to be consumed at any time based on table 1.

TABLE 1
Example 1.

| Component | Substance | g/L |
|---|---|---|
| Insoluble Active | Ivermectin | 6.000 |
| Solvent | Anhydrous Ethyl Alcohol | 100.000 |
| Sweetener | Sucralose | 8.000 |
| Flavor | Cherry | 10.000 |
| Antioxidant | Butylhydroxytoluene | 0.1000 |
| Vehicle | Medium Chain Triglycerides | 853.000 |

It should be taken into account that the non-aqueous solution formulation of Ivermectin should be taken before food.

The lipid medium proposed according to the present invention facilitates the absorption of water-insoluble active ingredients and allows the drug to be consumed at any time.

Only some preferred embodiments of the invention have been illustrated by way of example. In this regard, it will be appreciated that the formulation of Ivermectin in oral solution, as well as the configurative arrangements can be chosen from a plurality of alternatives without departing from the spirit of the invention according to the following claims.

### REFERENCES

Caly, L., Wagstaff, K. M., Jans, D. A., 2012. Nuclear trafficking of proteins from RNA viruses: potential target for anti-virals? Antivir. Res. 95, 202-206.
Frieman, M., et al., 2007. Severe acute respiratory syndrome coronavirus ORF6 antagonizes STAT1 function by sequestering nuclear import factors on the rough endoplasmic reticulum/Golgi membrane. J. Virol. 81 (18), 9812-9824.
Gonzalez Canga, A., et al., 2008. The pharmacokinetics and interactions of ivermectin in humans—a mini-review. AAPS J. 10 (1), 42-46.
Gotz, V., et al., 2016. Influenza A viruses escape from MxA restriction at the expense of efficient nuclear vRNP import. Sci. Rep. 6, 23138.
Hiscox, J. A., et al., 2001. The coronavirus infectious bronchitis virus nucleoprotein localizes to the nucleolus. J. Virol. 75 (1), 506-512.
Jans, D. A., Martin, A. J., Wagstaff, K. M., 2019. Inhibitors of nuclear transport. Curr. Opin. Cell Biol. 58, 50-60.
Kosyna, F. K., et al., 2015. The importin alpha/beta-specific inhibitor Ivermectin affects HIF-dependent hypoxia response pathways. Biol. Chem. 396 (12), 1357-1367.
Lundberg, L., et al., 2013. Nuclear import and export inhibitors alter capsid protein distribution in mammalian cells and reduce Venezuelan Equine Encephalitis Virus replication. Antivir. Res. 100 (3), 662-672.
Lv, C., et al., 2018. Ivermectin inhibits DNA polymerase UL42 of pseudorabies virus entrance into the nucleus and proliferation of the virus in vitro and vivo. Antivir. Res. 159, 55-62.
Mastrangelo, E., et al., 2012 August Ivermectin is a potent inhibitor of flavivirus replication specifically targeting NS3 helicase activity: new prospects for an old drug. J. Antimicrob. Chemother. 67 (8), 1884-1894.
Rowland, R. R., et al., 2005. Intracellular localization of the severe acute respiratory syndrome coronavirus nucleocapsid protein: absence of nucleolar accumulation during infection and after expression as a recombinant protein in vero cells. J. Virol. 79 (17), 11507-11512.
Tay, M. Y., et al., 2013. Nuclear localization of dengue virus (DENV) 1-4 non-structural protein 5; protection against all 4 DENV serotypes by the inhibitor Ivermectin. Antivir. Res. 99 (3), 301-306.
Timani, K. A., et al., 2005. Nuclear/nucleolar localization properties of C-terminal nucleocapsid protein of SARS coronavirus. Virus Res. 114 (1-2), 23-34.
van der Watt, P. J., et al., 2016. Targeting the nuclear import receptor Kpnbeta1 as an anticancer therapeutic. Mol. Canc. Therapeut. 15 (4), 560-573.
Wagstaff, K. M., et al., 2012. Ivermectin is a specific inhibitor of importin alpha/beta mediated nuclear import able to inhibit replication of HW-1 and dengue virus. Biochem. J. 443 (3), 851-856.
Wagstaff, K. M., et al., 2011. An AlphaScreen®-based assay for high-throughput screening for specific inhibitors of nuclear import. J. Biomol. Screen 16 (2), 192-200.
Wulan, W. N., et al., 2015. Nucleocytoplasmic transport of nucleocapsid proteins of enveloped RNA viruses. Front. Microbiol. 6, 553.
Wurm, T., et al., 2001. Localization to the nucleolus is a common feature of coronavirus nucleoproteins, and the protein may disrupt host cell division. J. Virol. 75 (19), 9345-9356.
Yang, S. N. Y., et al., 2020. The broad spectrum antiviral ivermectin targets the host nuclear transport importin alpha/beta1 heterodimer. Antivir. Res. 104760.

1-5. (canceled)

6. An Ivermectin formulation in solution suitable for oral administration which formulation comprises:
(a) effective amounts by weight of Ivermectin;
(b) 80% to 90% by weight of an oily solvent;
(c) 10% to 20% by weight of a co-solvent;
(d) 0.00001% to 0.1% by weight of an antioxidant; and
(e) flavorings and sweeteners.

7. The formulation of claim 6, wherein said effective amount of Ivermectin is an amount between 0.5 to 1.2% by weight.

8. The formulation of claim 6, wherein said oily solvent is selected from the group consisting of canola oil, corn oil, cottonseed oil, sesame oil, soybean oil and medium chain triglycerides.

9. The formulation of claim 8, wherein said medium chain triglycerides is selected from the group consisting of Caprylic/Capric Triglyceride and extracts from endosperms of palm oil and or coconut plants.

10. The formulation of claim 6, wherein said co-solvent is ethanol.

11. The formulation of claim 10, wherein said ethanol is anydrous ethanol.

12. The formulation of claim 6, wherein said antioxidant is selected from the group consisting of: butylhydroxyanisole (BHA), butylhydroxytoluene (BHT), propylgallate, sesamol, ascorbic acid, ascorbyl palmitate, malic acid, sodium ascorbate, sodium metabisulfite, tocopherol and DL-α-tocopherol.

13. The formulation of claim 12, wherein said anti-oxidant is butylhydroxytoluene.

14. The formulation of claim 12, wherein said antioxidant is butylhydroxyanisole.

15. The formulation of claim 12, wherein said antioxidant is DL-α-tocopherol.

16. The formulation of claim 12, wherein said antioxidant is ascorbic acid.

17. The formulation of claim 12, wherein said antioxidant is malic acid.

18. The formulation of claim 6, wherein said flavoring is cherry flavor.

19. The formulation of claim 6, wherein said sweetener is sucralose.

20. A rapidly absorbable Ivermectin formulation in solution suitable for oral administration which formulation comprises:
(a) effective amounts by weight of Ivermectin;
(b) 80% to 90% by weight of an oily solvent;
(c) 10% to 20% by weight of a co-solvent;
(d) 0.00001% to 0.1% by weight of an antioxidant; and
(e) flavorings and sweeteners.

21. The formulation of claim 20, wherein said effective amount of Ivermectin is an amount between 0.5 to 1.2% by weight.

22. The formulation of claim 20, wherein said oily solvent is selected from the group consisting of canola oil, corn oil, cottonseed oil, sesame oil, soybean oil and medium chain triglycerides.

23. The formulation of claim 20, wherein said medium chain triglycerides is selected from the group consisting of Caprylic/Capric Triglyceride and extracts from endosperms of palm oil and or coconut plants.

24. The formulation of claim 20, wherein said co-solvent is anhydrous ethanol.

25. The formulation of claim 20, wherein said antioxidant is selected from the group consisting of: butylhydroxyanisole (BHA), butylhydroxytoluene (BHT), propylgallate, sesamol, ascorbic acid, ascorbyl palmitate, malic acid, sodium ascorbate, sodium metabisulfite, tocopherol and DL-α-tocopherol.
